(19) United States
(12) Patent Application Publication
(10) Pub. No.: US 2021/0236841 A1
(43) Pub. Date: Aug. 5, 2021

(54) SYSTEM AND METHOD OF A HANDHELD LASER LIGHT PROBE

(71) Applicants: Coren Bruce, West Palm Beach, FL (US); Joseph Costello, West Palm Beach, FL (US)

(72) Inventors: Coren Bruce, West Palm Beach, FL (US); Joseph Costello, West Palm Beach, FL (US)

(21) Appl. No.: 16/783,031

(22) Filed: Feb. 5, 2020

(51) Int. Cl. A61N 5/06

(52) U.S. Cl. CPC A61N 5/0603; A61N 2005/0666; A61N 2005/067; A61N 2005/0608; A61N 2005/0611; A61N 2005/063

(57) ABSTRACT

A laser device for cancer treatment is provided comprising a handheld cylinder for partial insertion into a body cavity and a plurality of cone mirror reflectors in the cylinder, each reflector with a hollow central profile. The system further comprises a plurality of ring lenses in the cylinder and a subminiature (SMA) connector inside a first end of the cylinder that receives laser light from a fiber cable, the laser light then passing through the reflectors and the lenses and exiting the cylinder via a housing tip at a second end of the cylinder in a 360-degree arc. The cone mirror reflectors are alternatively solid with truncated apexes. The cylinder is made of one of borosilicate glass and polyamide resin.

### FIELD OF THE INVENTION

The present disclosure is in the field of healthcare devices. More particularly, systems and methods described herein provide a tubular device for insertion into body cavities that radiates laser light in a broad radius to cancerous tissues as well as assistance with pain management.

### BACKGROUND

Laser light radiation is known to successfully eliminate or reduce cancerous cells and tumors within the body's tissues and assist with pain management. Previous implementations in laser probes use single-angled reflectors or homogenous diffusers to emit a broken coverage over 360-degree radius.

Use of single angled reflectors may result in errant rays not striking the reflector face directly or reflecting at angles alternate to the desired angle of reflection. This undesired action may result in a loss of power with potential negative effects to the probe housing or worse. Further with the short focal lengths typically existing in laser probes used to treat body cavities, the divergence is minimal. The use must therefore be monitored in order to determine the effect of the laser light on the body's tissue. The objective is to ensure no unwanted damage is caused and to direct the laser light's treatment of the affected tissues.

In previous laser probe devices that use semi-invasive photochemical means for the treatment of cancerous body cavity tissues, there are either no membranes or only homogenous membranes between the patient's body tissue and the laser light. This may make hygiene a point to be addressed through lengthy sterilization methods.

Alternatively, the devices are made up of multiple parts to facilitate the insertion of the probe, but none have a sealed housing due to the known limitations of opto-mechanics and manufacturing challenges. Additionally, with profiled openings allowing the laser light to travel to the patient's tissue in the said probe devices, the potential for unwanted lacerations exist when the said probe comes into contact with the soft tissue of the body cavity, either during treatment or when being extracted from the body cavity.

### BRIEF DESCRIPTION OF THE DRAWINGS

FIG. 1 is a drawing of a handheld laser light probe in accordance with an embodiment of the present disclosure.

FIG. 2 is a drawing of a handheld laser light probe in accordance with an embodiment of the present disclosure.

FIG. 3 is a drawing of a handheld laser light probe in accordance with an embodiment of the present disclosure.

FIG. 4 is a drawing of a handheld laser light probe in accordance with an embodiment of the present disclosure.

FIG. 5 is a drawing of a handheld laser light probe in accordance with an embodiment of the present disclosure.

FIG. 6 is a drawing of a handheld laser light probe in accordance with an embodiment of the present disclosure.

FIG. 7 is a drawing of a handheld laser light probe in accordance with an embodiment of the present disclosure.

FIG. 8 is a drawing of a handheld laser light probe in accordance with an embodiment of the present disclosure.

### DETAILED DESCRIPTION

Systems and methods described herein provide a laser probe device which is tubular in form factor. The term “probe” or “device” as used herein covers the entire disclosure herein of a handheld device for insertion into body cavities. The device contains convex cone mirror reflectors that project laser light outward in a 360-degree arc. In addition to directing the laser light out in a 360-degree radius the light is diverged by the convex face on the convex cone mirror reflector. The laser light in a sense does pass through the cone reflector but it is also reflected off its convex reflective face. It is the unique hole in the center of the cone reflector that allows the light to pass through it to reflect of the reflector behind it and through its hole to the next reflector in line and so on. The device may be used to treat ailments inside body cavities, for example in endo-rectal or endo-vaginal treatments of cancerous tissues to reduce or eliminate such cancerous tissues. The device emits micro-pulsed 360-degree laser light from one end while receiving the laser light at the other end from an external power source. The laser light passes through the cone mirror reflectors and other optical elements that diverge the laser light as it travels outward into the patient's body.

There are three versions of the cone mirror reflectors with the “convex cone mirror reflector” being the primary embodiment. The cone mirror reflectors in the device enable reflection of a laser light beam equally in the 360-degree radius. This may eliminate problems associated with errant rays. Micro-pulsing of the laser light may reduce the risk of unwitting damage to the patient's body tissues. This may reduce the need for direct observation during the procedure. A practitioner may rely on stored data describing safe durations of heat source contact with tissues receiving stimulation.

The cone mirror reflectors may be provided with hollow central profiles in the reflectors, with solid reflector surfaces and with truncated apexes, or with a different structure. These structures promote laser light to pass by the first cone in the series and to reflect off subsequent cones along the linear path of the laser light. The subsequent cones reflect the laser light in the wide radius with minimal errant rays. The last conical mirror reflector in the series at a distal end of the probe reflects remaining laser light outward in the wide radius.

The probe connects to a high-powered class IV laser generator by means of a flexible fiber optic cable which is attached to the laser generator with a SMA905 (subminiature) connector and a Lemo connector in some embodiments. For purposes of this discussion, the proximal end of the tubular probe receives laser light from a fiber cable. The distal end is inserted into the patient. The fiber cable runs from the high-powered laser generator to the proximal end of the laser probe. At its proximal end it has a strain relief junction where it enters the probe to ensure the fiber cable is protected from extreme bending during repetitive use.

The fiber cable once past the strain relief and inside the proximal end of the probe ends at a SMA905 connector that is mounted into the probe's bulkhead by means of a SMA905 bulkhead adaptor. The fiber tip sits just inside the probe which extends away from a handle towards the distal end of the probe. Inside the probe are the four conical mirror reflectors which are seated inside the probe housing.

Along the length of the probe housing are ring lenses that allow the reflected laser light to pass outwards. In an all-glass embodiment of the probe where the probe itself is made from a high strength high heat glass such as borosilicate, the reflected laser light passes outward through the housing itself which acts as the optical medium. In the case of a polymer polyamide probe, the laser light is diffused by the probe housing itself. Reflected rays traveling back towards the fiber tip are reflected off an inverted cone mirror reflector with the fiber tip protruding through its center. Diverging beams will continue outwards from the probe housing. With the designs provided herein, maximum divergence may be achieved, in embodiments along most of the length of the probe. In all the designs with the exception of the Polymer polyamide version, approximately only two thirds of the probe tip are affected by the diverging laser light”. The solid polymer polyamide probe tip however would emit along its entire length due to the diffusion created by the particles throughout.

The probe with a hole through its center in many embodiments allows the laser light to pass by the first cone in the series to reflect off the subsequent cones placed along the laser lights' linear path. All the subsequent cones reflect the laser light in a 360-degree radius with few or no errant rays. The final conical mirror reflector in the series at the distal end of the probe reflects the remaining laser light outwards in a 360-degree radius thereby directing effectively all of the laser light in the desired direction.

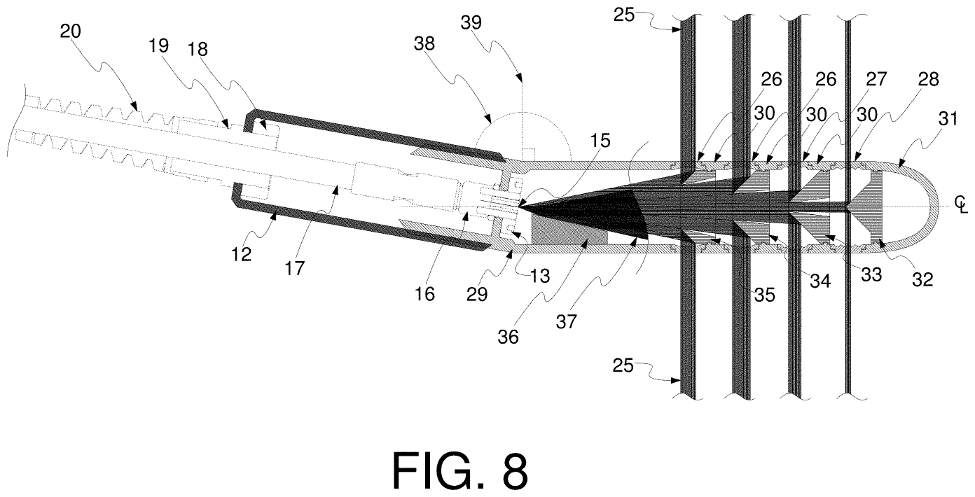

The probe is housed in a sealed glass unit with a smooth outer surface which may ensure that tissue is not lacerated. The ergonomic shape of the device allows laser light to enter the body cavity easily with the aid of lubricant. The sealed housing of the probe may support greater hygiene of the device. The device may have an outer sleeve that is discarded after each treatment. This sleeve may only be relevant for the segmented probe tip as illustrated in FIG. 8. The smooth probe tip (all glass version) does not need a protective disposable sleeve. As the device has a smooth surface, lesions are unlikely to occur in the sleeve. The exterior of the device may be made entirely of glass, facilitating sterilization.

Since the housing of the device is sealed, the laser light passes through an optical membrane or glass lens, further diverging the beam. This may allow for an effective difference to the timing of the micro-pulsing and the spread of the laser light. This may positively affect the areas covered by each micro-pulse. The effect of the divergence is noticeable as stated in the pulse times, only the method of divergence varies in each version with the all glass probe tip achieving little divergence while the ring lenses as illustrated in the angled version achieve a greater amount of divergence. The final divergence in the all glass version is achieved by the convex face of the cone mirror reflectors.

Concave lens faces, which in embodiments may be used, naturally diverge a light beam. The diameter or radius of the concave face determines the degree the divergence along with the focal length. A challenge with the divergence is that the diameter of the concave face is slight so it will most likely need to be similar to configurations where there are multiple rings as opposed to a single ring lens. By using a small diameter concave face, although be it a linear one will create a greater degree of divergence, and to place multiple rings of the small diameter faces next to one another one is creating a field of divergent rings, controlled by the size of the diameter used.

In primary embodiments, the cone mirror reflectors have a convex face which diverge the laser light beam path without the need for the ring lenses having any major divergence. The light beam reflects off the cone mirrors and outward at approximately a 90-degree angle from the probe tip.

In an embodiment, solid cone mirror reflectors are provided that only have the mirror coating applied to their conical faces. The rest of the mirror bodies are clear, allowing the light to pass through to the mirrors situated behind relative to the beams.

Cone mirror reflectors allow the light pass through them to the mirror behind, while the part of the mirror that reflects the light does so at 90 degrees outwards. The convex cone mirror reflector also diverges the beam.

While ring lenses are components herein, they may be of less importance than with concave mirrors because the divergence achieved using ring lenses is somewhat imperceptible. With convex cone mirror reflectors, the lack of divergence in the glass probe tip is addressed. With the four convex cone mirror reflectors in series, the beam should emit along at least a portion of the probe's length, diverging out from each cone mirror reflector.

The probe tip in many embodiments is made of borosilicate glass. A polyamide resin version of the probe tip is also provided and uses a quartzite particle fill. With the borosilicate glass option, the cone mirror reflectors may be secured in place without obscuring the probe front with any opaque materials. The ends of the borosilicate tubes are finished off to achieve the rounded shape for the probe. A thread is introduced on the handle side so it can be attached to the base (handle). In an embodiment, the cone mirror reflectors are captured within the homogeneous solid mass of the polymer polyamide material with no quartzite particles, but rather a clear polished mass with possibly a relevant coating.

In embodiments, the probe is provided via a telescoping assembly. An exploded perspective view of the probe is shown in the drawings provided herewith which depict the inner glass tubes that slide into the primary or outer glass tube. The inner glass tubes hold the convex cone mirror reflectors in place within the primary glass tube at the correct distances from the beam to achieve the optimum spread of the light beam.

While the probe is provided in many embodiments as a straight tube in borosilicate glass or other material, in embodiments the probe may be angled or slightly bent for ergonomic purposes. The principles are the same for both probes, being the manipulation of the laser lights path to its optimal output of a 360-degree radius beam along the length of the probe's tip with the maximum amount of divergence. The same or similar result is produced with both the straight and angled probe where the probe directs the laser lights path outwards at approximately a 90-degree angle from the laser lights original path with a great amount of divergence when it leaves the probe tip.

Turning to the figures, a listing of the components depicted in the figures is provided immediately below. Component numbering is consistent across the drawings. Not every component listed below appears in every drawing.
1. BOROSILICATE GLASS TIP HOUSING—OUTER
2. BOROSILICATE GLASS TIP HOUSING—INNER-A
3. BOROSILICATE GLASS TIP HOUSING—INNER-B
4. BOROSILICATE GLASS TIP HOUSING—INNER-C
5. BOROSILICATE GLASS TIP HOUSING—INNER-D
6. BOROSILICATE GLASS TIP HOUSING—INNER-E
7. CONVEX CONE MIRROR REFLECTOR—1
8. CONVEX CONE MIRROR REFLECTOR—2
9. CONVEX CONE MIRROR REFLECTOR—3
10. CONVEX CONE MIRROR REFLECTOR—4
11. SMA-ADAPTER
12. HOUSING-HANDLE
13. SMA905 BULKHEAD ADPATER
14. SMA905 BULKHEAD ADAPTER NUT
15. FIBER TIP
16. SMA905 CONNECTOR
17. FIBER OPTIC CABLE
18. STRAIN RELIEF ASSEMBLY NUT
19. STRAIN RELIEF ASSEMBLY BOLT
20. STRAIN RELIEF ASSEMBLY FLEXIBLE END
21. SWITCH ADAPTER RING
22. SWITCH
23. SWITCH MOUNTING NUT
24. DETAIL EXTENTS
25. LASER LIGHT BEAM EXTENTS
26. RING LENS-TYPE 1
27. RING LENS-TYPE 2
28. RING LENS-TYPE 3
29. ANGLED SMA ADAPTER-BASE
30. SMA ADAPTER RING HOUSING
31. SMA ADAPTER TIP
32. CONE MIRROR REFLECTOR—1
33. CONE MIRROR REFLECTOR—2
34. CONE MIRROR REFLECTOR—3
35. CONE MIRROR REFLECTOR—4
36. ANGLED MIRROR REFLECTOR
37. REFLECTED LASER LIGHT OFF ANGLED MIRROR REFLECTOR 36
38. ANGLE OF PROBE HANDLE FROM LINE DEFINING THE 90 DEGREE ANGLE
39. LINE DEFINING THE 90 DEGREE ANGLE OFF THE PROBE TIP

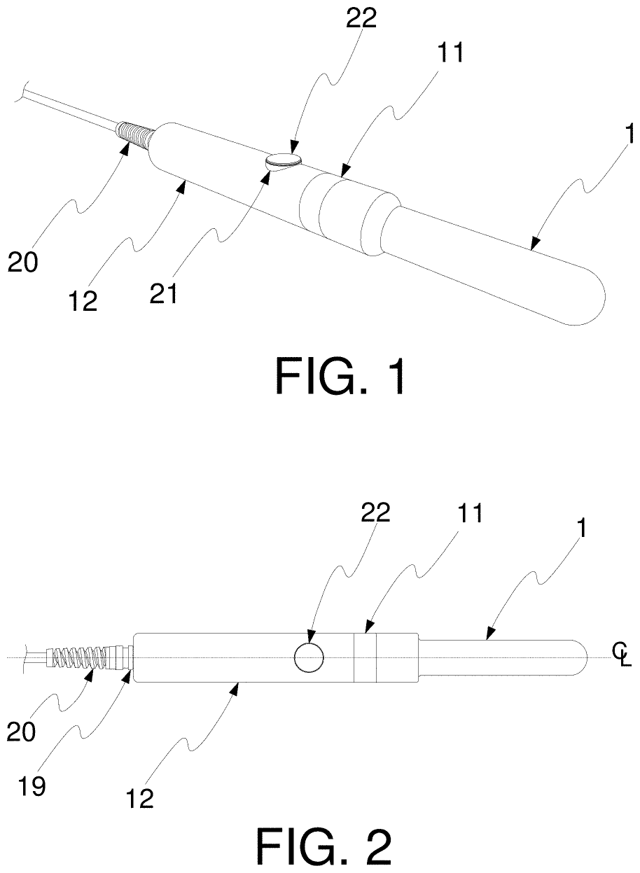

FIG. 1 is a diagram of a handheld laser light probe in accordance with an embodiment of the present disclosure. FIG. 1 provides a simple perspective view of the probe with the borosilicate glass tip housing—outer 1 partially insertable into a body cavity of a patient. On the far left of the device is a fiber cable leading to a laser generator providing laser light. The laser generator is not shown in the drawings provided herein.

FIG. 2 is a diagram of a handheld laser light probe in accordance with an embodiment of the present disclosure. FIG. 2 provides a top view of the probe. The overlapped capital letters “C” and “L” depicted in FIG. 2 indicate an invisible centerline of the probe.

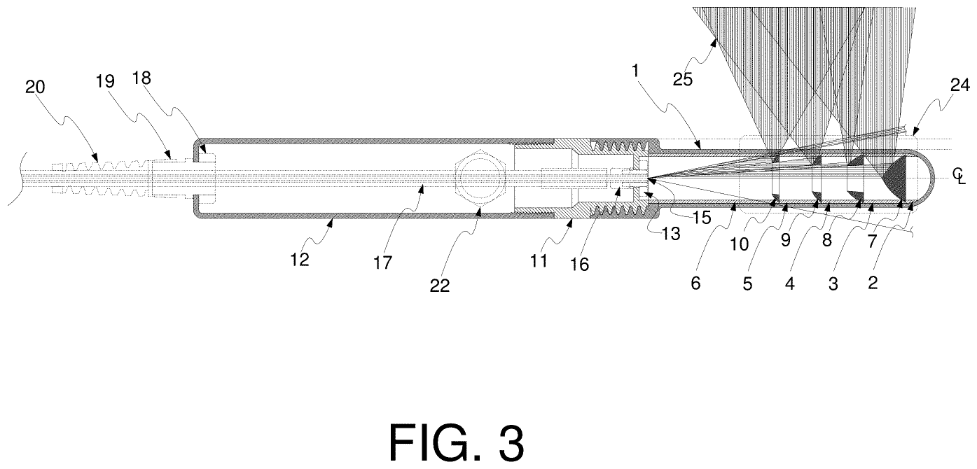

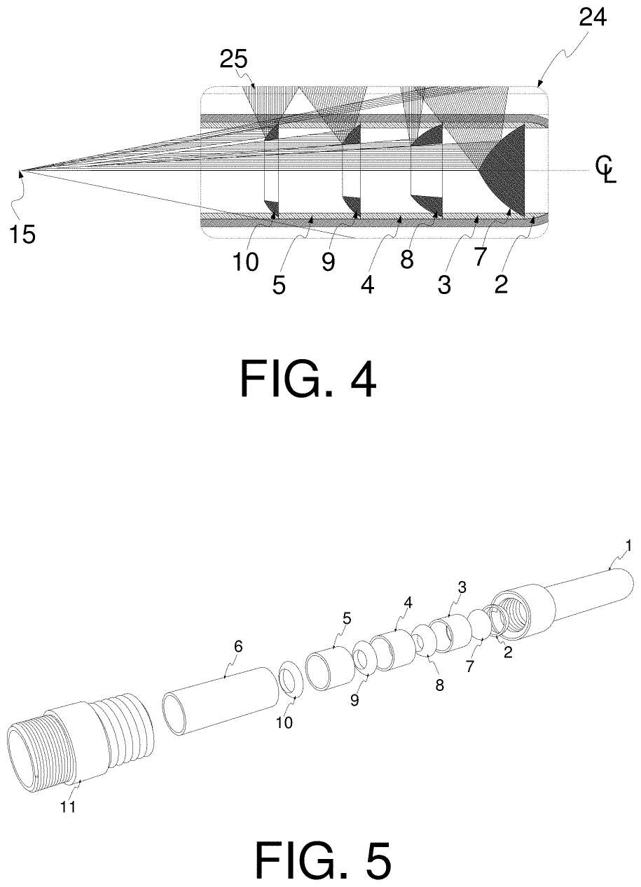

FIG. 3 is a more detailed view of the probe with the laser light shown projecting from the sides of the probe. This is a section view of the probe with the section line running along the center axis of the probe when viewed from the side. The convex cone mirror reflectors are shown reflecting the laser light from the sides of the probe at broad angles to expose to affected areas of the patient's tissue to the laser. FIG. 4 is a close-up view of some of the components depicted in FIG. 3. The laser light being projected outward is shown in detail. The laser light is only shown from one side in at least FIGS. 3 and 4 to make viewing more practical while in reality the laser light would emit in a full 360-degree radius.

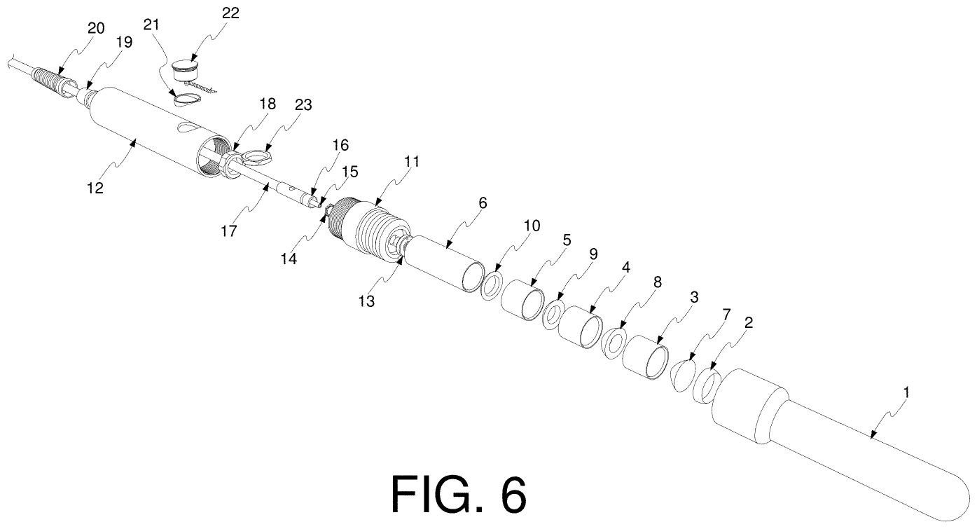

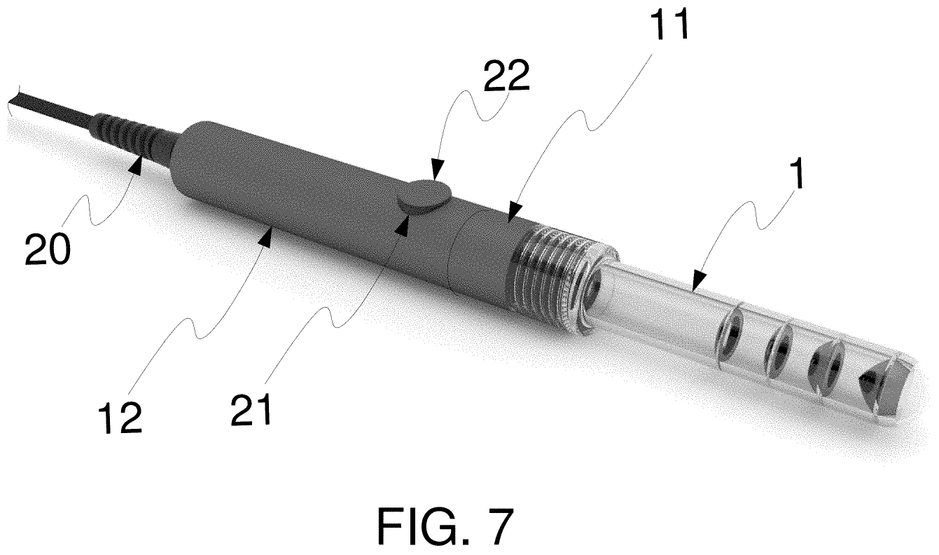

FIG. 5 is an exploded perspective view showing the borosilicate glass parts, the convex cone mirror reflectors 7-10 and the SMA adapter 11 viewed from the back. FIG. 6 is an exploded perspective view showing the entire probe assembly viewed from the side front. FIG. 7 is a perspective view render of the probe viewed from the side front.

As noted, the probe may be provided in a slightly angled form factor for ergonomic reasons. FIG. 8 is a diagram of the probe in the angled form factor.

Two additional ring lens designs are included in the angled version in the interest of achieving greater divergence. They are ring lens-type 2 27 and ring lens-type 3 28. Ring lens-type 1 26 is the original ring lens design and would work in conjunction with the convex cone mirror reflectors to achieve the desired divergence. The principle of ring lens-type 3 28 is based on known physical attributes of optical properties being that a concave face naturally diverges. Systems and methods use the multiple rings with concave faces based on known optical principals that a concave face diverges the light path. By using a small diameter concave face, although be it a linear one will create a greater degree of divergence, and to place multiple rings of the small diameter faces next to one another one is creating a field of divergent rings, controlled by the size of the diameter used.

Ring lens-type 2 27 is a further development with the small concave rings radiating out towards the lenses' outer face with a view to diverging the beam closer to the lens face thereby improving the ultimate divergence.

The angled mirror reflector 36 is the angled flat mirror reflector redirecting the primary laser light towards the cone mirror reflectors 1-4 with reflected laser light off angled mirror reflector (36) 37 being the redirected beam. The sma adapter 11 has been relabeled component 29 in the angled probe, although its function is substantially identical to the SMA adapter 11 in that it is the adapter between the handle which contains the switch and the probe front or tip which contains the cone mirror reflectors and the ring lenses. It is different in that it is angled, hence the new number allocation.

The angled probe is also made up of the SMA adapter ring housings 30 which hold the cone mirror reflectors 1-4 in place. The cone mirror reflectors now have new numbers, 32, 33, 34, and 35 as they are different in shape and therefore will have a different effect on the beam. In the angled probe design the cone mirror directs the beam to the ring lenses which in turn diverge the beam, while in the straight probe design the convex cone mirror reflectors diverge the beam themselves.

The principles are substantially alike for both the straight and the angled probes. The laser light's path is directed to its optimal output of a 360-degree radius beam along the length of the probes tip with the maximum amount of divergence. In both the straight and angled designs, elements can be adapted from either design. Either principle may operate in either of the two embodiments, being a straight probe or the angled probe with the same or similar result where a laser probe directs the laser lights path outwards at approximately a 90-degree angle from the laser lights original path with a great amount of divergence when it leaves the probe tip.

1. A laser device for cancer treatment, comprising:
a handheld cylinder for partial insertion into a body cavity;
a plurality of cone mirror reflectors in the cylinder, each reflector with a hollow central profile;
a plurality of ring lenses in the cylinder; and
a subminiature (SMA) connector inside a first end of the cylinder that receives laser light from a fiber cable, the laser light then passing through the reflectors and the lenses and exiting the cylinder via a housing tip at a second end of the cylinder in a 360-degree arc.

2. The system of claim 1, wherein the cone mirror reflectors are alternatively solid with truncated apexes.

3. The system of claim 1, wherein the cylinder is made of one of borosilicate glass and polyamide resin.

4. The system of claim 1, wherein the housing tip is inserted into a body cavity.

5. The system of claim 1, wherein the laser light distributed in the 360-degree arc is directed to cancerous tissues in the body cavity.

6. The system of claim 1, wherein the ring lenses have concave faces.

7. The system of claim 1, wherein the fiber cable receives the laser light from a high-power laser generator.

8. The system of claim 1, wherein the device is one of straight and angled.

9. The system of claim 1, wherein the laser light is micro-pulsing.

10-14. (canceled)

15. A method for applying laser light to internal body cavity areas, comprising:
a handheld cylinder receiving laser light via a subminiature connector at a first end of the cylinder;
the cylinder channeling the light through a plurality of ring lenses and a plurality of cone mirror reflectors, the light diverging during said channeling; and
the cylinder, via a housing tip, channeling the light outward in a 360-degree arc,
further comprising an SMA connector receiving the laser light via fiber cable connector with a high-powered laser generator external to the cylinder,
further comprising the cylinder diverging the laser light based on a convex curved profile of the cone mirror reflectors,
further comprising the cylinder receiving partial insertion into a body cavity of a patient,
further comprising the cylinder directing the laser light to internal tissues near the body cavity, and
further comprising the light passing through the cone mirror reflectors via one of hollow central profiles in the reflectors and solid reflector surfaces with truncated apexes.

16-20. (canceled)
